(19) United States
(12) Patent Application Publication
(10) Pub. No.: US 2008/0280981 A1
(43) Pub. Date: Nov. 13, 2008

(54) Tranexamic acid formulations

(75) Inventors: Keith A. Moore, Loveland, OH (US); Ralph A. Heasley, Union, KY (US); Jeffrey S. Greiwe, Ft. Thomas, KY (US)

Correspondence Address: Davidson, Davidson & Kappel, LLC, 485 7th Avenue, 14th Floor, New York, NY, 10018, US

(73) Assignee: Xanodyne Pharmaceuticals, Inc., Florence, KY (US)

(21) Appl. No.: 12/220,241

(22) Filed: Jul. 23, 2008

Related U.S. Application Data
(63) Continuation of application No. 11/072,162, filed on Mar. 4, 2005.
(60) Provisional application No. 60/550,113, filed on Mar. 4, 2004.
(60) Provisional application No. 60/592,885, filed on Jul. 30, 2004.

(51) Int. Cl. A61K 31/195; A61P 7/04

(52) U.S. Cl. 514/561

(57) ABSTRACT

Disclosed are oral tranexamic acid formulations and methods of treatment therewith.

This application is a continuation of U.S. patent application Ser. No. 11/072,162 filed Mar. 4, 2005 which claims the benefit of U.S. Provisional Patent Application No. 60/550,113, filed Mar. 4, 2004, and U.S. Provisional Patent Application No. 60/592,885, filed Jul. 30, 2004 the disclosures of which is are hereby incorporated by reference in their entirety entireties.

### FIELD OF THE INVENTION

The invention is directed to oral tranexamic acid formulations and methods of treatment with these formulations.

### BACKGROUND OF THE INVENTION

Tranexamic acid (trans-4-(aminomethyl)cyclohexanecarboxylic acid, Cyklokapron® (Pfizer) is an antifibrinolytic agent. That is, it helps to prevent lysis or dissolution of a fibrin clot which forms in the normal physiologic process of hemostasis. Its mechanism of action is as a competitive inhibitor of plasminogen activation, and as a noncompetitive inhibitor of plasmin; both plasminogen and plasmin are activators of fibrinolysis and active clot-lysing agents. Tranexamic acid thus helps to stabilize fibrin clots, which in turn maintains coagulation and helps to control bleeding.

Tranexamic acid is used to control excess bleeding, for example, excess bleeding that occurs during dental procedures in hemophiliacs and for heavy bleeding during menstruation (menorrhagia). Women suffering from menorrhagia are typically treated orally with 500 mg tranexamic acid tablets administered three or four times daily with a total daily dose ranging from 3 grams/day (two tablets every eight hours) to 6 grams/day (three tablets every six hours). However, this treatment may cause adverse gastrointestinal reactions, including nausea, vomiting, diarrhea, and cramping, etc. These gastrointestinal side effects are due to the quantity of tranexamic acid and/or rapid rate of release of tranexamic acid into the stomach with each dose, as well as the large quantity of excipients used in the tablet formulation that are introduced into the stomach. Such side effects, in addition to the cramping, bloating, pain, and other symptoms that may accompany menses, are undesirable, and a formulation of tranexamic acid is needed which will reduce or eliminate these side effects.

### SUMMARY OF THE INVENTION

It is an object of the invention to provide an oral dosage form comprising tranexamic acid which is suitable for oral administration on a two or three times a day basis to humans.

It is a further object of certain embodiments of the invention to provide a method of treating a patient in need of tranexamic acid therapy with one or more oral dosage forms comprising tranexamic acid.

It is a further object of certain embodiments of the present invention to provide a method of treating a patient suffering from heavy menstrual bleeding (menorrhagia) by orally administering to the patient one or more dosage forms comprising tranexamic acid which provide(s) for therapeutically effective levels of tranexamic acid suitable for two or three times a day administration.

It is a further object certain embodiments of the invention to provide a formulation comprising an amount of tranexamic acid which when released in the gastric contents provides fewer adverse effects than with certain current tranexamic acid therapy.

The above advantages and objects and others can be achieved by virtue of the present invention which is directed in part to a method of treating a patient in need of tranexamic acid therapy comprising administering to the patient about 1300 mg of tranexamic acid or pharmaceutically acceptable salt thereof in one or more oral dosage forms including the tranexamic acid or pharmaceutically acceptable salt thereof such that the dose administered provides a mean maximum plasma concentration (Cₘₐₓ) of tranexamic acid of from about 9 to about 15 mcg/ml, preferably from about 10 to about 14 mcg/ml, more preferably about 12 mcg/ml after single dose oral administration to humans.

In certain embodiments, the invention is directed to an oral dosage form comprising 650 mg of tranexamic acid or pharmaceutically acceptable salt thereof which provides a mean maximum plasma concentration (Cₘₐₓ) of tranexamic acid of from about 9 to about 15 mcg/ml, preferably from about 10 to about 14 mcg/ml, more preferably about 12 mcg/ml per 1300 mg of tranexamic acid after single dose oral administration to humans.

In certain embodiments, the invention is directed to an oral dosage form comprising 650 mg of tranexamic acid or pharmaceutically acceptable salt thereof which provides a mean Tₘₐₓ of tranexamic acid at from about 2 to about 4 hours, preferably at about 3 hours after oral administration to humans.

In certain embodiments, the invention is directed to an oral dosage form comprising about 650 mg of tranexamic acid or pharmaceutically acceptable salt thereof and a pharmaceutically acceptable excipient; said dosage form providing an in-vitro dissolution release rate of the tranexamic acid, when measured by a USP 27 Apparatus Type II Paddle Method @ 50 RPM in 900 ml water at 37±0.5° C., of at least about 70% by weight tranexamic acid or pharmaceutically acceptable salt thereof or released at about 45 minutes, and preferably 100% by weight tranexamic acid or pharmaceutically acceptable salt thereof release by about 45 minutes.

In certain embodiments, the invention is directed to an oral dosage form comprising about 650 mg of tranexamic acid or pharmaceutically acceptable salt thereof and a pharmaceutically acceptable excipient, said dosage form providing an in-vitro dissolution release rate of the tranexamic acid or pharmaceutically acceptable salt thereof from the dosage form, when measured by the USP 27 Apparatus Type II Paddle Method @ 50 RPM in 900 ml water at 37±0.5° C. of about 0% to about 95% by weight tranexamic acid or pharmaceutically acceptable salt thereof released at about 15 minutes, from about 30% to about 100% by weight tranexamic acid or pharmaceutically acceptable salt thereof released at about 30 minutes, from about 70% to about 100% by weight tranexamic acid or pharmaceutically acceptable salt thereof released at about 45 minutes, about 100% by weight tranexamic acid or pharmaceutically acceptable salt thereof released at about 60 minutes.

In certain embodiments, the invention is directed to an oral dosage form comprising about 650 mg of tranexamic acid or pharmaceutically acceptable salt thereof and a pharmaceutically acceptable excipient, said dosage form providing an in-vitro dissolution release rate of the tranexamic acid or pharmaceutically acceptable salt thereof from the dosage form, when measured by the USP 27 Apparatus Type II Paddle Method @50 RPM in 900 ml water at 37±0.5° C. of about 50% to about 95% by weight tranexamic acid or pharmaceutically acceptable salt thereof released at about 15 minutes, from about 70% to about 100% by weight tranexamic acid or pharmaceutically acceptable salt thereof released at about 30 minutes, from about 80% to about 100% by weight tranexamic acid or pharmaceutically acceptable salt thereof released at about 45 minutes, about 100% by weight tranexamic acid or pharmaceutically acceptable salt thereof released at about 60 minutes.

In certain embodiments, the invention is further directed to an oral dosage form comprising tranexamic acid (preferably in an amount of about 650 mg) or pharmaceutically acceptable salt thereof and a pharmaceutically acceptable excipient, which provides for a bioavailability of tranexamic acid of greater than 40%, from about 41% to about 60%, preferably from about 42% to about 50%, more preferably about 46% after oral administration to humans.

In certain embodiments, the invention is further directed to an oral dosage form comprising about 650 mg of tranexamic acid or pharmaceutically acceptable salt thereof and a pharmaceutically acceptable excipient, the dosage form being suitable for oral administration on a twice a day basis, and the dosage form providing a mean maximum plasma concentration (Cₘₐₓ) of tranexamic acid of from about 13.5 to about 22.5 mcg/ml, preferably from about 15 to about 21 mcg/ml, more preferably about 18 mcg/ml per 1950 mg tranexamic acid after single dose oral administration to humans.

In certain embodiments, the invention is further directed to a method of treating a patient with a therapeutically effective amount of tranexamic acid or pharmaceutically acceptable salt thereof comprising administering to the patient two or three dosage forms of the present invention, each dosage form comprising from about 585 mg to about 715 mg of tranexamic acid or pharmaceutically acceptable salt thereof, preferably about 650 mg tranexamic acid or pharmaceutically acceptable salt thereof, and a pharmaceutically acceptable excipient.

In certain embodiments, the invention is directed to a dose of tranexamic acid or pharmaceutically acceptable salt thereof comprising two oral dosage forms, each oral dosage form comprising from about 585 mg to about 715 mg, preferably about 650 mg of tranexamic acid or pharmaceutically acceptable salt thereof, and a pharmaceutically acceptable excipient, the dose providing a therapeutic effect when administered three times a day.

In certain embodiments, the invention is directed to a dose of tranexamic acid or pharmaceutically acceptable salt thereof comprising three oral dosage forms, each oral dosage form comprising from about 585 mg to about 715 mg, preferably about 650 mg of tranexamic acid or pharmaceutically acceptable salt thereof, and a pharmaceutically acceptable excipient, the dose providing a therapeutic effect when administered twice a day.

In certain preferred embodiments, the oral dosage form of the invention further provides a mean transit time of said tranexamic acid of 7.21±1.01 hours when administered across a patient population.

In certain preferred embodiments, the oral dosage form of the invention further provides a mean absorption time of said tranexamic acid of 3.70±0.94 hours when administered across a patient population.

In certain embodiments, the invention is further directed to an oral dosage form comprising tranexamic acid or pharmaceutically acceptable salt thereof and at least one pharmaceutically acceptable excipient which provides less than about 20 percent incidence of nausea, less than about 15 percent incidence of nausea, preferably less than about 10 percent incidence of nausea as a side effect after single dose oral administration across a patient population.

In certain embodiments the dosage form provides less CNS side effects (e.g., headache), less GI side effects (e.g., nausea), or combination thereof in comparison to a therapeutically equivalent amount of tranexamic acid administered intravenously in five minutes or less when administered across a patient population.

In certain preferred embodiments, the therapeutically effective dose of the tranexamic acid is provided via the administration of two or more dosage units. For example, if the dosage unit comprises 650 mg of tranexamic acid and the dose for administration is about 1300 mg then two dosage units would be administered to a patient in need of such treatment, or for example, when the dose for administration is 1950 mg, three dosage units would be administered.

In certain preferred embodiments, the invention is further directed to a method of treating a patient with one or more oral dosage forms comprising tranexamic acid or pharmaceutically acceptable salt thereof and a pharmaceutically acceptable excipient, wherein the oral dosage form provides a therapeutically effective plasma level of tranexamic acid in accordance with a three times a day (TID) dosing schedule, and the therapeutically effective dose administered comprises about 1300 mg of tranexamic acid.

In certain preferred embodiments, the invention is further directed to a method of treating a patient with one or more oral dosage forms comprising tranexamic acid or pharmaceutically acceptable salt thereof and a pharmaceutically acceptable excipient, wherein the oral dosage form provides a therapeutically effective plasma level of tranexamic acid in accordance with a twice a day (BID) dosing schedule, and the therapeutically effective dose administered comprises about 1950 mg of tranexamic acid.

In certain embodiments, the tranexamic acid for use in the methods and formulations of the present invention is in the form of a pharmaceutically acceptable salt thereof. Such salt forms include for example and without limitation the sodium salt, potassium salt, calcium salt, magnesium salt and the like; as well as the hydrochloride, hydrobromide, sulfate, phosphate, formate, acetate, trifluoroacetate, maleate, tartrate methanesulfonate, benzenesulfonate, p-toluenesulfonatemethanesulfonate salt forms, and the like. Preferably the active ingredient for use in accordance with the present invention is tranexamic acid.

The term “Cₘₐₓ” unless otherwise indicated is meant for purposes of the present invention to mean the maximum plasma concentration of a medicament achieved after single dose administration of a dosage form, or the maximum plasma concentration of a medicament achieved over a dosing interval from multiple-doses at steady-state in accordance with the present invention.

The term “Tₘₐₓ” is meant for purposes of the present invention to mean the elapsed time from administration of a dosage form to the time the Cₘₐₓ of the medicament is achieved.

The term “steady state” means that the amount of the drug reaching the system is approximately the same as the amount of the drug leaving the system. Thus, at “steady-state”, the patient's body eliminates the drug at approximately the same rate that the drug becomes available to the patient's system through absorption into the blood stream.

The term “mean” for purposes of the present invention, when used to define a pharmacokinetic value (e.g., Tₘₐₓ), unless specified otherwise, represents the arithmetic mean value measured across a patient or subject population.

The term “three times a day (TID) basis” for purposes of the present invention, means that the dosage regimen is to be administered three times a day, preferably on a schedule of every 8 hours.

The term “mean transit time” is understood by those skilled in the art and means the time-point where 63.2% of the total AUC is attained after oral administration, or 63.2% of the IV dose is eliminated, as described in Applied Pharmacokinetics, Principles of Therapeutic Drug Monitoring, Second Edition (1986), edited by William E. Evans, et al., the disclosure of which is hereby incorporated by reference in its entirety.

The term “mean absorption time” is understood by those skilled in the art and means a quantitative parameter which summarizes how long, on average, the drug molecule remains unabsorbed, i.e. persists in its dosage form and GI tract, also as described in Applied Pharmacokinetics, Principles of Therapeutic Drug Monitoring, Second Edition (1986), edited by William E. Evans, et al. Its value is not affected like the drug's absorption rate constants (ka), which can be skewed, due to incomplete release of drug from its dosage form, irregular absorption, lag-time, mixed zero-order dissolution rates, changing GI motility, GI blood flow, first-pass effect, etc.

### BRIEF DESCRIPTION OF THE FIGURES

FIG. 1 depicts mean plasma concentration-time profiles on a semi-log scale over 36 hours for the study of Example 3.

FIG. 2 depicts mean plasma concentration-time profiles on a linear scale over 36 hours for the study of Example 3.

### DETAILED DESCRIPTION

The dosage regimen typically listed for tranexamic acid in HMB (Heavy Menstrual Bleeding) therapy is 1-1.5 g per dose administered three-four times a day at the onset of copious menstrual bleeding and continued for the first 3-5 days of the menstrual cycle. However, the most frequently reported dosage regimen of tranexamic acid is an immediate release oral formulation in which 1 g tranexamic acid is administered four times a day (4 g per day) for HMB therapy outside of the US. Knowledge of this common regimen is supported by a careful review of the randomized controlled trials published in the medical literature, product labeling from other countries' regulatory authorities having the product approved for HMB therapy, utilization data from Sweden (Rybo 1991), correspondence and interviews with non-US clinicians having experience with the product. That regimen is currently the dosage being studied by the US Center for Disease Control (CDC) in women with HMB associated with bleeding disorders.

The absolute bioavailability of tranexamic acid observed when administering the European commercial formulation (Cyklokapron, KAbi AB, Sweeden Batch 90288; assay 499 mgm/tablet) to male subjects is approximately 35% and its elimination correlates with renal creatinine clearance. Peak serum tranexamic acid concentrations occur approximately 3 hours after the oral administration of a European immediate-release tablet formulation (>85% dissolved at 15 minutes) (Pilbrant, et al., Eur. J. Clin. Pharmacol, (1981)-20:65-72). By comparison, the in vivo absorption profile observed with the European immediate-release formulation is slow and very gradual over 3 hours. Specifically, tranexamic acid serum concentrations are 9, 41, 73, 88 percent (with food), and 22, 63, 85, and 98 percent (fasting) of maximal absorption at 0.5, 1, 1.5 and 2 hours after a 2 g oral dose, respectively. Although not wishing to be held to any specific theory, it is presently hypothesized that tranexamic acid oral absorption appears to be controlled by a non-dissolution rate limited process, i.e. the rate and extent of oral absorption is a function of a transmembrane passage-limited process, in order to explain the disparity between the time of product dissolution and relatively prolonged tmax (time to achieve the peak serum concentration).

Preferably, the goal of the formulation, dose strength and dosage regimen of the invention, is to provide HMB therapy which achieves from about 20% to 100% reduction in menstrual blood loss per menstrual cycle.

In accordance with the present invention an oral dosage form comprising about 650 mg of tranexamic acid is disclosed. Preferably, the oral dosage form contains at least one pharmaceutically acceptable excipient.

In certain embodiments, the oral dosage form of the present invention provides for an increased bioavailability as compared to other immediate release oral dosage forms currently available (e.g., Cyclokapron). In certain preferred embodiments the increased bioavailability allows therapeutic plasma levels of tranexamic acid to be reached with a lower dose of drug. Preferably, the increased bioavailability also decreases the amount of tranexamic acid that remains unabsorbed in the gastrointestinal which leads to decreased incidence of side effects that are typically associated with formulations that provide higher levels of unabsorbed tranexamic acid and prolonged exposure of the gastrointestinal tract to the higher tranexamic acid levels. Preferably the oral dosage form of the present invention provides for a bioavailability of tranexamic acid of greater than 40%, from about 41% to about 60%, preferably from about 42% to about 50%, more preferably about 46% after oral administration to humans.

In certain embodiments, the invention is directed to a method of providing a tranexamic acid plasma concentration within the range of about 5 mcg/mL to about 15 mcg/mL by administration of at least one formulation of the present invention comprising tranexamic acid and pharmaceutically acceptable excipient on a three times a day basis to a patient in need of tranexamic acid treatment.

In certain embodiments, the invention is further directed to a method of treating a human patient with heavy menstrual bleeding (e.g., menorrhagia) comprising administering about 1300 mg of tranexamic acid on a three times a day basis to the human patient to provide a tranexamic acid plasma concentration within the range of about 5 mcg/mL to about 15 mcg/mL after steady state oral administration to a human patient.

In certain embodiments, the invention is directed to a method of treating a patient suffering from menorrhagia, conization of the cervix, epistaxis, hyphema, hereditary angioneurotic edema, a patient with a blood coagulation disorder undergoing dental surgery, combinations thereof, and the like, by administering at least one dosage form of the present invention to the patient in need in tranexamic acid therapy.

In certain embodiments, the invention is directed to a method of treating heavy menstrual bleeding with a therapeutically effective dose of at least one oral formulation of the present invention comprising tranexamic acid and a pharmaceutically acceptable excipient wherein the menstrual blood loss per menstrual cycle is reduced by at least about 10 ml, preferably at least about 20 ml, more preferably at least about 40 ml. In a most preferred embodiment the menstrual blood loss per menstrual cycle is reduced by greater than or equal to about 50 ml.

In certain embodiments, the invention is directed to a method of treating heavy menstrual bleeding with a therapeutically effective dose of at least one oral formulation of the present invention comprising tranexamic acid and a pharmaceutically acceptable excipient which upon oral administration to a human female reduces the blood loss per menstrual cycle by about 35 ml to about 200 ml, preferably about 40 ml to about 175 ml, more preferably from about 50 ml to about 150 ml.

In certain embodiments, the invention is further directed to a method of treating heavy menstrual bleeding with a therapeutically effective dose of at least one oral formulation of the present invention comprising tranexamic acid and a pharmaceutically acceptable excipient which upon oral administration to a human female reduces the blood loss per menstrual cycle by about 20% to 100%, preferably from about 20% to about 70%.

The menstrual blood loss can be measured by procedures known in the art. For example, in certain embodiments, the menstrual blood loss can be determined by a procedure described by (i) L. Hallbert, et al. in “Determination of Menstrual Blood Loss”, Scandinav. J. Clin. & Lab. Investigation 244-248, 16, 1964, wherein the procedure is performed by extracting the menstrual blood from vaginal tampons and towels with a sodium hydroxide solution, converting heme chromogens to alkaline hematin, which is determined spectrophotometrically; or (ii) the menstrual blood loss can be determined by a procedure described by J. Newton, M.D., et al., in “A Rapid Method for Measuring Menstrual Blood Loss Using Automatic Extraction.”, Contraception, 269-282, September 1977, Vol. 16, No. 3, wherein the procedure is based upon the formation of alkaline haematin after the blood has been extracted from vaginal tampons and sanitary towels by an automatic Stomacher Lab-Blender. The disclosures of the aforementioned articles are hereby incorporated by reference in their entireties.

The oral dosage forms of the present invention may be prepared as tablets, capsules, granules, pellets, powders, pellets, dragees, troches, non-pariels, pills or encapsulated suspension, and may be packaged into capsules, sachets, etc.

Tranexamic acid oral dosage forms of the present invention are formulated to provide about a 650 mg dose of tranexamic acid. Typically, two oral dosage forms are administered to a patient in need of tranexamic acid therapy to provide a total dose of about 1300 mg. In addition, at least one pharmaceutically acceptable excipient is included in the oral dosage form. The pharmaceutically acceptable excipient may include, for example and without limitation, preservatives, diluents (e.g., microcrystalline cellulose), lubricants (e.g., stearic acid, magnesium stearate, and the like), binders (e.g., povidone, starch, and the like), disintegrants (e.g., croscarmellose sodium, corn starch, and the like), glidants (e.g., talc, colloidal silicon dioxide, and the like), granulating aids, colorants, and flavorants that are conventional in the pharmaceutical art. Specific examples of pharmaceutically acceptable excipients that may be used to formulate oral dosage forms are described in the Handbook of Pharmaceutical Excipients, American Pharmaceutical Association (2003), incorporated by reference herein.

Examples of diluents include dextrose, sucrose, starch, powdered cellulose, lactose, mannitol, microcrystalline cellulose, combinations thereof, and the like.

Examples of lubricants include magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, zinc stearate, combinations thereof and the like.

Examples of disintegrants include crospovidone, alginates, cellulose and its derivatives, clays, polyvinylpyrrolidone, polysaccharides, such as corn and potato starch, dextrins, croscarmellose sodium, sugars, combinations thereof, and the like.

Binders, when added to the formulation, promote granulation and/or promote cohesive compact during the direct compression into tablets. Examples of binders include povidone, acacia, cellulose derivatives, gelatin, glucose, polyvinylpyrrolidone, sodium alginate and alginate derivatives, sorbitol, starch, combinations thereof, and the like.

As used herein, alleviation of adverse effects using these formulations indicates any relief in one or more symptoms, such as decrease in incidence, severity, or duration of symptoms, and is not limited to absence of symptoms or elimination of symptoms. Thus, treatment includes any decrease in incidence, duration, intensity, frequency, etc. of adverse gastrointestinal symptoms including, but not limited to, headache, nausea, vomiting, diarrhea, constipation, cramping, bloating, and combinations thereof. The formulations may reduce symptoms at any time during tranexamic acid therapy, but minimized adverse effects are particularly noted immediately or shortly after dosing, that is, within the first few hours after dosing. As used herein, adverse gastrointestinal effects and side effects are used interchangeably to indicate nontherapeutic effects (i.e., not relating to any possible beneficial effects due to tranexamic acid), ranging from unpleasant but tolerable sensations to severe gastrointestinal symptoms. As used herein, the terms oral formulations, ingestable formulations, and orally administered formulations are used interchangeably and include any dosage forms which are ingested by mouth, including, but not limited to, tablets, pills, liquids, gelcaps, dragees, capsules, powders, granules, pellets, etc.

In certain embodiments, the formulation includes tranexamic acid in the range of about 50% by weight to about 95% or more by weight of the formulation. In other embodiments, tranexamic acid is in the range of about 60% by weight to about 90% by weight, or about 60% by weight to about 80% by weight of the formulation. The remaining weight may be made-up of the pharmaceutically acceptable excipient.

To prepare formulations of the present invention, the pharmaceutically acceptable excipient may be incorporated into e.g., a tablet matrix with the tranexamic acid or coated onto e.g., the tranexamic acid or both. In certain embodiments, tablet formulations prepared are formulated by granulating a blend of powders of the pharmaceutically acceptable excipient and the tranexamic acid. The powder blend is formed by combining portions of the powdered components that make up the tablet. These powders are intimately mixed by dry-blending. The dry blended mixture is granulated by wet mixing of a solution of a binding agent with the powder blend. Following granulation, the particles are removed from the granulator and placed in a fluid bed dryer, a vacuum dryer, a microwave dryer, or a tray dryer for drying. Drying conditions are sufficient to remove unwanted granulating solvent, typically water, or to reduce the amount of granulating solvent to an acceptable level. Drying conditions in a fluid bed dryer or tray dryer are typically about 50 to 70° C. The granulate is dried, screened, mixed with additional excipients such as disintegrating agents, flow agents, or compression aids and lubricants such as talc, stearic acid, or magnesium stearate, and compressed into tablets.

In certain embodiments, the tablet may be coated with an optional film-forming agent. This applied film may aid in identification, mask an unpleasant taste, allow desired colors and surface appearance, provide enhanced elegance, aid in swallowing, etc. The amount of film-forming agent may be in the range of about 2% tablet weight to about 4% tablet weight. Suitable film-forming agents are known to one skilled in the art and include hydroxypropyl cellulose, cellulose ester, cellulose ether, one or more acrylic polymer(s), hydroxypropyl methylcellulose, cationic methacrylate copolymers (diethylaminoethyl)methacrylate/methyl-butyl-methacrylate copolymers such as Eudragit E® (Rohm Pharma) and the like. The film-forming agents may optionally contain colorants, plasticizers, fillers, etc. including, but not limited to, propylene glycol, sorbitan monooleate, sorbic acid, titanium dioxide, and one or more pharmaceutically acceptable dye(s).

In certain embodiments, tranexamic acid tablets are formulated by dry blending, rotary compacting, or wet granulating powders composed of tranexamic acid and tablet excipients. These powders are compressed into a tablet.

Alternatively, the tranexamic acid formulations may be administered by pellets or granules in e.g., a sachet or capsule. Tranexamic acid pellets or granules may be prepared by using the pharmaceutically acceptable excipient to form a granule or pellet matrix. U.S. Pat. Nos. 5,650,174; and 5,229,135 each of which is expressly incorporated by reference herein in its entirety, disclose variations on fabricating a pellet or nonpareil dosage form. Spheres are filled into packets, termed sachets, or capsules which are filled by weight to contain the prescribed dose of drug.

In certain embodiments, the tranexamic acid formulations of the present invention may be used for additional indications other than menorrhagia, such as conization of the cervix, epistaxis, hyphema, hereditary angioneurotic edema, a patient with a blood coagulation disorder undergoing dental surgery, combinations thereof, and the like.

### DETAILED DESCRIPTION OF PREFERRED EMBODIMENTS

The invention will be further appreciated with respect to the following non-limiting examples. Other variations or embodiments of the invention will also be apparent to one of ordinary skill in the art from the above descriptions and examples. Thus, the forgoing embodiments are not to be construed as limiting the scope of this invention.

### Example 1

In Example 1, immediate release 650 mg tranexamic acid tablets were prepared having the ingredients listed in Table 1 below:

TABLE 1

| | Quantity | |
|---|---|---|
| | per batch | Quantity per |
| Ingredient | (kg) | tablet (mg) |
| Active Ingredient | | |
| Tranexamic Acid, EP (650 mg/tab) | 84.50 | 650.0 |
| Inactive Ingredients | | |
| Microcrystalline Cellulose, NF | 5.753 | 44.25 |
| (Avicel PH 101) | | |
| Microcrystalline Cellulose, NF | 10.660 | 82.00 |
| (Avicel PH 102) | | |
| Colloidal Silicon Dioxide, NF | 0.0975 | 0.75 |
| Pregelatinized Corn Starch, NF | 6.435 | 49.50 |
| Croscarmellose Sodium, NF | 19.50 | 15.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water, USP* | 17.550 | 135.00 |
| Film Coating (Inactive Ingredients)** | | |
| Opadry White YS-1-7003 | 4.110 | — |
| Purified Water, USP | 36.990 | — |

*Purified water is removed during processing
**6 kg excess prepared to account for losses during transfer

The formulation of Example 1 was prepared as follows:
1. Weigh all ingredients and keep in moisture resistant containers until ready for use.
2. Measure water into a container. Mix povidone at medium speed until completely dissolved.
3. Add tranexamic acid, microcrystalline cellulose (MCC), pregelatinized corn starch, and colloidal silicon dioxide to the high shear mixer.
4. Mix using impeller only.
5. Mix for an additional time (impeller only). Add all of the povidone solution during this mixing step.
6. Mix until adequately granulated (impeller and chopper). Proceed only when desired granulation has been achieved. Add additional water if necessary.
7. Dry the granulation to moisture content of NMT 1.2%.
8. Pass the granulation through the oscillating granulator equipped with a #30 mesh screen. Weigh the granulation. Add granulation to the V-Blender.
9. Add the croscarmellose sodium and MCC to the V-Blender and blend.
10. Pass magnesium stearate and stearic acid through oscillating granulator equipped with a #40 mesh screen. Add magnesium stearate and stearic acid to the V-blender and blend.
11. Perform specified physical property testing. Proceed to compression.
12. Compress tablets.
12. After compression, spray coat the compressed dosage forms with the Opadry White in water.

### Example 2

Modified release 650 mg tranexamic acid tablets were prepared having the ingredients listed in the Table 2 below:

TABLE 2

| | Quantity | Quantity |
|---|---|---|
| | per batch | per tablet |
| Ingredient | (kg) | (mg) |
| Active Ingredient | | |
| Tranexamic Acid, EP | 84.50 | 650.0 |
| Inactive Ingredients | | |
| Microcrystalline Cellulose NF (Avicel PH 101) | 5.753 | 44.25 |
| Colloidal Silicon Dioxide NF | 0.0975 | 0.75 |
| Pregelatinized Corn Starch, NF | 6.435 | 49.50 |
| Hypromellose, USP (Methocel K3 Premium LV) | 19.110 | 147.00 |
| Povidone, USP (K value range 29-32) | 4.680 | 36.00 |
| Stearic Acid, NF (powder) | 2.340 | 18.00 |
| Magnesium Stearate, NF (powder) | 0.585 | 4.50 |
| Purified Water USP* | 17.550 | 135.00 |

*Purified water is removed during processing

The formulation of Example 2 was prepared as follows:
1. Weigh all ingredients and keep in moisture resistant containers until ready for use.
2. Measure water into a container. Mix povidone at medium speed until completely dissolved.
3. Add tranexamic acid, microcrystalline cellulose (MCC), pregelatinized corn starch, and colloidal silicon dioxide to the high shear mixer.
4. Mix using impeller only.
5. Mix for an additional time (impeller only). Add all of the povidone solution during this mixing step.
6. Mix until adequately granulated (impeller and chopper). Proceed only when desired granulation has been achieved. Add additional water if necessary.
7. Dry the granulation to moisture content of NMT 1.2%.
8. Pass the granulation through the oscillating granulator equipped with a #30 mesh screen. Weigh the granulation. Add granulation to the V-Blender.
9. Add the hypromellose USP Methocel K3 Premium to the V-blender. Blend.
10. Pass magnesium stearate and stearic acid through oscillating granulator equipped with a #40 mesh screen. Add magnesium stearate and stearic acid to the V-blender and blend.
11. Perform specified physical property testing. Proceed to compression.
12. Compress tablets to desired weight.

### Example 3

### Bioavailability and Bioequivalence Evaluation

In Example 3, a comparative, randomized, single dose, 4-way Crossover Absolute Bioavailability (BA) and Bioequivalence (BE) study of Tranexamic Acid Tablet Formulations prepared in accordance with Examples 1 and 2 in Healthy Adult Women Volunteers under Fasting Conditions was performed. The objective was to assess the bioequivalence of a 650 mg immediate release tablet formulation prepared in accordance with Example 1 compared to the modified release tablet formulation of tranexamic acid prepared in accordance with Example 2, and to determine the bioavailability of the tablet formulations to the approved IV (1 g) formulation Cyklokapron® by Pharmacia & Upjohn. The design was a randomized, 4-way crossover, comparative BE and BA determination. All oral doses administered were 1.3 g. Twenty-eight (28) healthy non-smoking adult female volunteer subjects were enrolled in the study. Sample size was calculated assuming a 25% CV in AUC_{inf}. The study endpoints were the 90% confidence intervals of the ratio of least-squares means of the pharmacokinetic parameters AUC₀₋ₜ, AUC_{inf} and Cₘₐₓ of the modified release formulation to the immediate-release formulation from serum concentration-time data drawn up to 36 hours after a single dose of drug. In addition, the bioavailability of the tablet formulations were calculated. Smokers, oral contraceptive users, those with a previous history of thromboembolic events and altered vision were excluded from the study. ECG monitoring was performed before, during and after the estimated times of peak serum tranexamic acid concentrations exposure. Adverse events were captured and recorded throughout the trial period.

In the study, subjects were randomized to receive single oral 1.3 g (2×650 mg tablets) dose of tranexamic acid in tablet forms which included a modified release dosage form and an immediate release dosage form. Subjects were also administered a single 1 g (10 ml) IV solution of tranexamic acid (100 mg/ml concentration).

A summary of the pharmacokinetic results from the study of Example 3 are listed in the tables below.

TABLE 3
Summary of Results - Tranexamic Acid in Plasma
Pharmacokinetic Parameters
(N = 26)

| | ln AUC 0-t* | ln AUCinf* | ln Cmax* |
|---|---|---|---|
| | (mcg · h/mL) | (mcg · h/mL) | (mcg/mL) |
| Modified Release | | | |
| formulation | | | |
| Mean | 66.703 | 69.642 | 11.251088 |
| CV | 26.8 | 27.2 | 29.1 |
| N | 26 | 24 | 26 |
| Immediate Release | | | |
| formulation | | | |
| Mean | 70.157 | 72.656 | 12.260414 |
| CV | 16.2 | 16.4 | 23.0 |
| N | 26 | 24 | 26 |
| Least-Squares Mean: | | | |
| Modified Release | 66.935 | 68.891 | 11.321919 |
| Immediate Release | 70.051 | 72.411 | 12.258222 |
| Ratio of | 95.6 | 95.1 | 92.4 |
| Least-Squares Mean | | | |
| (modified | | | |
| release/immediate | | | |
| release)% | | | |

*For ln-transformed parameters, the antilog of the mean (i.e. the geometric mean) is reported. AUCinf, kel, half-life and F could not be estimated for some subjects.

TABLE 4
Summary of Results - Tranexamic Acid in Plasma
Pharmacokinetic Parameters
(N = 26)

| | Tmax | Half-life | kel | F |
|---|---|---|---|---|
| | (h) | (h) | (l/h) | (%) |
| Modified | | | | |
| Release | | | | |
| formulation | | | | |
| Mean | 2.942 | 11.370 | 0.06300 | 44.93 |
| CV | 22.7 | 17.6 | 19.4 | 25.3 |
| n | 26 | 26 | 26 | 24 |
| Immediate | | | | |
| Release | | | | |
| formulation | | | | |
| Mean | 2.808 | 11.013 | 0.06438 | 46.04 |
| CV | 20.8 | 15.5 | 15.3 | 16.1 |
| n | 26 | 24 | 24 | 24 |

TABLE 5
Summary of Results - Tranexamic Acid in Plasma
Pharmacokinetic Parameters
(N = 26)

| | Ln AUC 0-t* | ln AUCinf* | ln Cmax* |
|---|---|---|---|
| | (mcg · h/mL) | (mcg · h/mL) | (mcg/mL) |
| 90% Confidence | | | |
| Intervals | | | |
| (Modified | | | |
| release/Immediate | | | |
| release)% | | | |
| lower limit: | 87.8% | 87.4% | 84.0% |
| upper limit: | 104.0% | 103.5% | 101.6% |
| p-Value (ANOVA) | | | |
| Modified vs Immediate | 0.3721 | 0.3259 | 0.1676 |
| Period | 0.0704 | 0.0499 | 0.0356 |
| Sequence | 0.7734 | 0.7978 | 0.8207 |
| Intrasubject CV % | 18.3 | 17.4 | 20.6 |

*For ln-transformed parameters, the antilog of the mean (i.e. the geometric mean) is reported. AUCinf, kel, half-life and F could not be estimated for some subjects. AUC 0-t is the area under the plasma concentration versus time curve, from time 0 to the last measurable concentration, as calculated by the linear trapezoidal method.

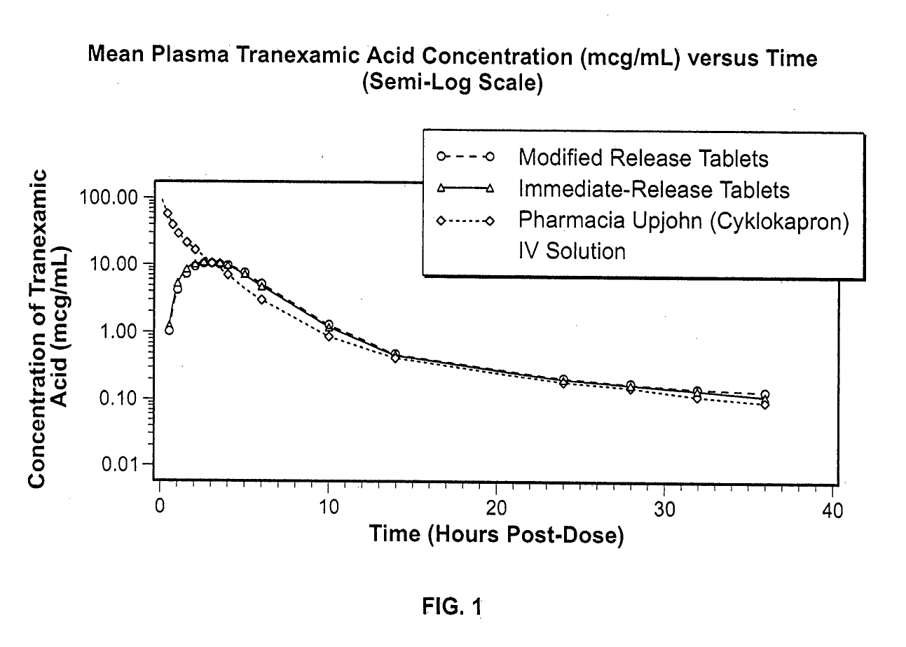

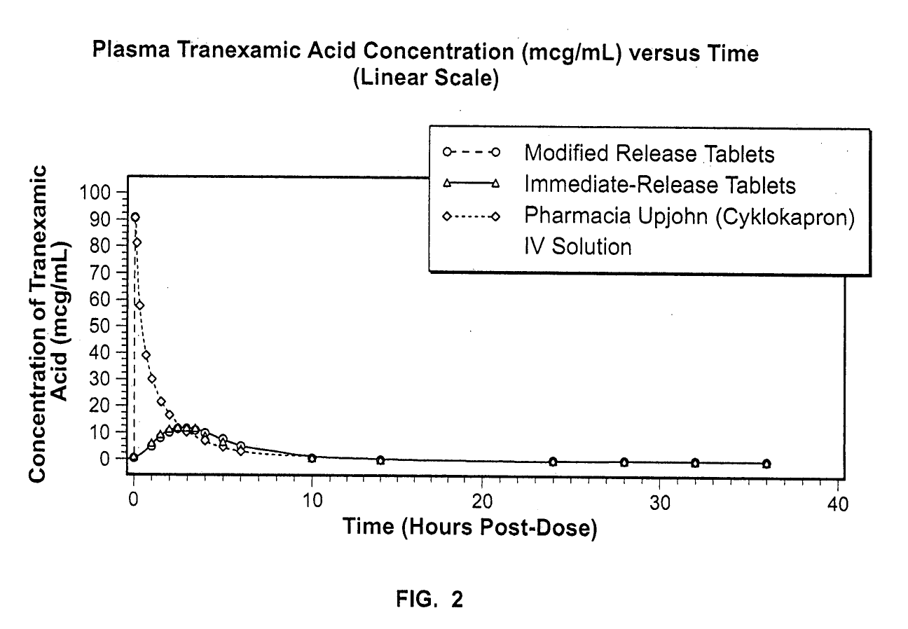

Concentration-time profiles for the study of Example 3 are presented on semi-log and linear scale over 36 hours and are depicted in FIGS. 1 and 2.

The following pharmacokinetic parameters in the table below were calculated for tranexamic acid in plasma for the study of Example 3.
MRT: The mean residence time (MRT) after intravenous administration of tranexamic acid was determined using the equation,

AUMC/AUC+infusion time/2,
where the AUMC is the area under the moment-time curve.
MTT: Following oral administration of the Modified Release and Immediate Release formulations, the mean transit time (MTT) of tranexamic acid was calculated by dividing the AUMC by the AUC.
MAT: The mean absorption time (MAT) for the two formulations was derived by subtracting the MRT from the MTT.
Mean (±SD) results are presented in the table below:

TABLE 5

| | IV | Modified Release | Immediate Release |
|---|---|---|---|
| MRT (hours) | 3.51 ± 0.38 | N/A | N/A |
| MTT (hours) | N/A | 7.70 ± 0.72 | 7.21 ± 1.01 |
| MAT (hours) | N/A | 4.18 ± 0.70 | 3.70 ± 0.94 |

The mean transit time (MTT) and mean absorption time (MAT) of the Modified Release formulation of tranexamic acid was approximately 30 minutes longer than that observed for the Immediate Release formulation.

The most frequently reported adverse events from the study of Example 3 are listed in the table below. The table lists the number of subjects reporting adverse events, and the percentage of subjects is in parentheses.

TABLE 6

| | Treatment | | |
|---|---|---|---|
| | Modified | Immediate | |
| | Release | Release | IV solution |
| | (2 × 650 mg) | (2 × 650 mg) | (10 × 100 mg/ml) |
| Adverse Events | (n = 27) | (n = 27) | (n = 27) |
| Headache | 4 (15%) | 7 (26%) | 7 (26%) |
| Nausea | 0 (0%) | 2 (7%) | 10 (37%) |
| Dizziness | 0 (0%) | 0 (0%) | 11 (41%) |
| Feeling Hot | 0 (0%) | 0 (0%) | 6 (22%) |
| Nasal Congestion | 2 (7%) | 1 (4%) | 1 (4%) |
| Cough | 0 (0%) | 0 (0%) | 2 (7%) |
| Urine odor abnormal | 2 (7%) | 0 (0%) | 1 (4%) |

Dissolution Results for Immediate Release and Modified Release Formulations prepared in accordance with Examples 1 and 2 respectively used in the study of Example 3 tested under USP 27 Apparatus Type II Paddle Method @ 50 RPM in 900 ml water at 37±0.5° C. are listed in the tables below.

TABLE 7
Test Results for the Immediate Release Formulation in Table 1.

| | % | RSD |
|---|---|---|
| Assay | 99.9% | |
| Content Uniformity | 99.4% | 0.7% |
| Unknown Related | NMT 0.2% Each | <0.1% |
| Substance | | |
| Total Related Substances | NMT 2.0% Total | <0.1% |
| and Impurities | | |
| Dissolution Profile | | |
| 15 min. | 58.0% | |
| 30 min. | 96.0% | |
| 45 min. | 102.0% | |
| 60 min. | 104.0% | |

TABLE 8
Test Results for the Modified Release Formulation in Table 2

| | % | RSD |
|---|---|---|
| Assay | 99.4% | |
| Content Uniformity | 98.5% | 0.6% |
| Unknown Related | NMT 0.2% Each | <0.1% |
| Substance | | |
| Total Related Substances | NMT 2.0% Total | <0.1% |
| and Impurities | | |
| Dissolution Profile | | |
| 15 min. | 21.0% | |
| 30 min. | 40.0% | |
| 45 min. | 58.0% | |
| 60 min. | 73.0% | |
| 90 min. | 98.0% | |

### CONCLUSIONS

The ratios of least-squares means and the 90% confidence intervals derived from the analyses of the In-transformed pharmacokinetic parameters AUC₀₋ₜ, AUC_{inf} and Cₘₐₓ for tranexamic acid in plasma were within the 80-125% Food and Drug Administration (FDA) acceptance range for the modified release formulation versus the immediate release formulation under fasting conditions.

The absolute bioavailability of the modified release and immediate release tablet formulations were 44.93% and 46.04% respectively.

Based on these results, the modified release tranexamic acid tablet formulation and the immediate release tranexamic acid formulation are bioequivalent under fasting conditions.

While the invention herein disclosed has been described by means of specific embodiments and applications thereof, numerous modifications and variations could be made thereto by those skilled in the art without departing from the spirit and scope of the present invention. Such modifications are understood to be within the scope of the appended claims.

What is claimed is:

1. A method of treating a patient in need of tranexamic acid therapy comprising:
orally administering to said patient about 1300 mg of tranexamic acid or pharmaceutically acceptable salt thereof in at least one oral dosage form which provides a mean maximum plasma concentration (Cₘₐₓ) of tranexamic acid of about 9 to about 15 mcg/ml after single dose oral administration to humans.

2. The method of claim 1, wherein said at least one oral dosage form provides a mean time to maximum plasma concentration (Tₘₐₓ) at about 2 to about 4 hours after oral administration to humans.

3. The method of claim 1, wherein the dosage form provides a mean transit time of said tranexamic acid of 7.21±1.01 hours.

4. The method of claim 1, wherein the oral dosage form of claim 1, wherein the dosage form provides a mean absorption time of said tranexanic acid of 3.70±0.94 hours.

5. The method of claim 1, wherein said at least one oral dosage form is two oral dosage forms.

6. The method of claim 1, wherein said patient is a patient suffering from menorrhagia, conization of the cervix, epistaxis, hyphema, hereditary angioneurotic edema, a patient with a blood coagulation disorder undergoing dental surgery, or combination thereof.

7. A oral dosage form comprising about 650 mg of tranexamic acid or pharmaceutically acceptable salt thereof and a pharmaceutically acceptable exicipient; said dosage form providing an in-vitro dissolution release rate of the tranexamic acid or pharmaceutically acceptable salt thereof, when measured by a USP 27 Apparatus Type II Paddle Method @ 50 RPM in 900 ml water at 37±0.5° C., of at least about 70% by weight tranexamic acid released at about 45 minutes.

8. The oral dosage form of claim 7, which provides an in-vitro dissolution release rate of the tranexamic acid or pharmaceutically acceptable salt thereof, when measured by the USP 27 Apparatus Type II Paddle Method @ 50 RPM in 900 ml water at 37±0.5° C. of about 0% to about 100% by weight tranexamic acid or pharmaceutically acceptable salt thereof released at about 15 minutes, from about 30% to about 100% by weight tranexamic acid or pharmaceutically acceptable salt thereof released at about 30 minutes, from about 70% to about 95% by weight tranexamic acid or pharmaceutically acceptable salt thereof released at about 45 minutes, about 100% by weight tranexamic acid or pharmaceutically acceptable salt thereof released at about 60 minutes.

9. The oral dosage form of claim 7, wherein the dosage form provides a mean transit time of said tranexamic acid of 7.21±1.01 hours when orally administered across a patient population.

10. The oral dosage form of claim 7, wherein the dosage form provides a mean absorption time of said tranexamic acid of 3.70±0.94 hours when orally administered across a patient population.

11. The oral dosage form of claim 7, wherein the pharmaceutically acceptable excipient comprises a diluent.

12. The oral dosage form of claim 11, wherein said diluent is selected from the group consisting of dextrose, sucrose, starch, powdered cellulose, lactose, mannitol, microcrystalline cellulose, and combinations thereof.

13. The oral dosage form of claim 7, wherein the pharmaceutically acceptable excipient comprises a glidant, a surface active agent, a coloring agent, a flavoring agent, a lubricant, or combination thereof.

14. A method of treating a patient in need of tranexamic acid therapy comprising: orally administering to said patient two dosage forms, each dosage form comprising about 650 mg of tranexamic acid or pharmaceutically acceptable salt thereof and a pharmaceutically acceptable excipient.

15. The method of claim 14, wherein the dosage forms provide a dissolution release rate in-vitro of the tranexamic acid or pharmaceutically acceptable salt thereof, when measured by the USP 27 Apparatus Type II Paddle Method @ 50 RPM in 900 ml water at 37±0.5° C., of least about 70% by weight tranexamic acid released at about 45 minutes.

16. The method of claim 14, wherein the dosage forms are administered to said patient three times a day.

17. The method of claim 14, wherein the pharmaceutically acceptable excipient comprises a diluent.

18. The method of claim 17, wherein said diluent is selected from the group consisting of dextrose, sucrose, starch, powdered cellulose, lactose, mannitol, microcrystalline cellulose, and combinations thereof.

19. The method of claim 14, wherein the pharmaceutically acceptable excipient comprises a glidant, a surface active agent, a coloring agent, a flavoring agent, a lubricant, or combination thereof.

20. An oral dosage form comprising tranexamic acid or a pharmaceutically acceptable salt thereof and a pharmaceutically acceptable excipient, said dosage form providing a bioavailability of said tranexamic acid of greater than 40%.

21. The oral dosage form of claim 20, wherein the dosage form comprises about 650 mg of said tranexamic acid or pharmaceutically acceptable salt thereof.

22. The oral dosage form of claim 21, which provides a dissolution release rate in-vitro of the tranexamic acid or pharmaceutically acceptable salt thereof, when measured by the USP 27 Apparatus Type II Paddle Method @ 50 RPM in 900 ml water at 37±0.5° C., of at least about 70% by weight tranexamic acid released at about 45 minutes.
